(19) United States
(12) Patent Application Publication
(10) Pub. No.: US 2004/0171691 A1
(43) Pub. Date: Sep. 2, 2004

(54) Propofol with cysteine

(75) Inventors: Hua Tang, Newton, MA (US); Hongming Chen, Acton, MA (US); Orn Almarsson, Shrewsbury, MA (US)

Correspondence Address: Transform Pharmaceuticals, Inc., 29 Hartwell Avenue, Lexington, MA, 02421, US

(73) Assignee: Transform Pharmaceuticals, Inc., Lexington, MA

(21) Appl. No.: 10/695,291

(22) Filed: Oct. 28, 2003

Related U.S. Application Data
(60) Provisional application No. 60/422,196, filed on Oct. 29, 2002.

(51) Int. Cl. A61K 31/198; A61K 31/05

(52) U.S. Cl. 514/562; 514/731

(57) ABSTRACT

The present invention relates to pharmaceutical compositions comprising 2,6-diisopropylphenol (propofol). Compositions of the present invention comprise aqueous and non-aqueous compositions of propofol and cysteine or a salt thereof. The propofol containing compositions are preferably sterile and are parenterally administered to any animal, including humans.

### RELATED APPLICATIONS

This application claims benefit of prior U.S. Provisional Patent Application No. 60/422,196 filed Oct. 29, 2002.

### BACKGROUND OF THE INVENTION

The compound 2,6-diisopropylphenol (propofol) is a well-known anesthetic agent. The onset of anesthesia is largely controlled by a drug's diffusion rate through the blood-brain barrier. Propofol is lipophilic and this helps the compound to provide rapid anesthetic action. However, this lipophilicity renders propofol, which is a liquid at room temperature, relatively insoluble in water. As a result, propofol is commonly administered (directly into the bloodstream either by infusion or by bolus injection) as an oil-in-water emulsion, containing a lipid component. Lipids, however, are good substrates for bacterial growth.

Despite the shortcomings of oil-in-water emulsions, propofol has been a successful anesthetic and is commercially available as Diprivan® Injectable Emulsion (AstraZeneca; Diprivan® is a trademark of Imperial Chemical Industries PLC) for human administration. Propofol is also marketed for veterinary use as Rapinovet™ Anesthetic Injection (Schering-Plough Animal Heath Corp.; Rapinovet™ is a trademark of Schering-Plough Veterinary Corp.) and as PropoFlo™ Anesthetic Injection (Abbott Laboratories; PropoFlo™ is a trademark of Abbott Laboratories).

Diprivan® Injectable Emulsion is a white, oil-in-water emulsion containing, in addition to 10 milligrams propofol per milliliter of emulsion, 100 mg soybean oil/mL, 22.5 mg glycerol/mL, 12 mg egg lecithin/mL, 0.005% disodium edetate, and sodium hydroxide. Diprivan® Injectable Emulsion is indicated as a single-use parenteral product. Diprivan® contains disodium edetate to retard the growth of microorganisms in the event of extrinsic contamination. Diprivan®, however, can still support the growth of microorganisms. As acknowledged in the product insert, there have been reports in which failure to use antiseptic technique when handling the emulsion was associated with microbial contamination and associated medical complications. Tubing and unused portions of Diprivan® should be discarded after 12 hours because of the potential for microbial growth. Diprivan® must be stored in the narrow temperature range of 4 to 22° C. (Diprivan® Injectable Emulsion Product Insert, AstraZeneca (2001)).

PropoFlo™ Anesthetic Injection is an oil-in water emulsion containing, in addition to 10 milligrams propofol per milliliter of emulsion, 100 mg soybean oil/mL, 22.5 mg glycerol/mL, 12 mg egg lecithin/mL, and sodium hydroxide. Like Diprivan®, PropoFlo™ is capable of supporting the growth of microorganisms. Failure to follow aseptic procedures may result in microbial contamination and associated medical complications. Unused portions of PropoFlo™ should be disposed of within 6 hours of vial entry. (PropoFlo™ Anesthetic Injection Product Insert, Abbott Laboratories (1998)).

Rapinovet™ Anesthetic Injection is a white, oil-in-water emulsion containing, in addition to 10 milligrams propofol per milliliter of emulsion, 100 mg soybean oil/mL, 22.5 mg glycerol/mL, 12 mg egg lecithin/mL, 0.25 mg sodium metabisulfite/mL, and sodium hydroxide. Like Diprivan® and PropoFlo™, Rapinovet™ is capable of supporting the growth of microorganisms. (Rapinovet™ Anesthetic Injection Product Insert, Schering-Plough Animal Health (2000)).

A need exists for non-toxic stable propofol formulations containing excipients which limit bacterial growth.

### SUMMARY OF THE INVENTION

The present invention relates to pharmaceutical compositions comprising 2,6-diisopropylphenol (i.e., propofol) or a prodrug of propofol and cysteine or a salt thereof. Compositions of the present invention comprise aqueous and non-aqueous formulations, including but not limited to, oil in water and water in oil emulsions. The propofol containing compositions are preferably sterile and are parenterally administered to any animal, including humans.

The instant invention is directed to several propofol-containing compositions as described below.

In one embodiment, the invention is directed to a composition comprising propofol, cysteine, and one or more excipients. Excipients can be any GRAS excipient. Examples of excipients include, but are not limited to, purified poloxamer, Ammonium acetate, Benzalkonium chloride, Benzethonium chloride, Benzyl alcohol, Brij 35, Brij 97, Calcium gluceptate, ChlorobutanOL, Citric Acid, Cremophor EL, Deoxycholate, Diethanolamine, Ethanol, Gamma cyclodextrin, Glycerin, Lactobionic acid, Lysine, Magnesium chloride, Methylparaben, PEG 1000, PEG 300, PEG 3350, PEG 400, PEG 600, Poloxamer 188, Poloxamer 237, Poloxamer 338, Poloxmer 407, Polyoxyethylene 100 stearate, Polyoxyethylene 40 stearate, Polyoxyethylene 50 stearate, Polysorbate 20, Polysorbate 80, Povidone, Propylene Glycol, Sodium acetate, Vitamine E TPGS, Sodium benzoate, Sodium tartate, vegetable oil, soy bean oil, safflower oil, cottonseed oil, corn oil, sunflower oil, arachis oil, castor oil, olive oil, an ester of a medium or long-chain fatty acid, a palmitate, a glyceral ester, polyoxyl hydrogenated castor oil, ethoxylated ethers, polypropylene-polyethylene block co-polymers, phosphatides, egg phosphatide, soy phosphatide, glycerin, ascorbic acid and gentisic acid, and monosodium glutamate.

In another embodiment, the composition comprises an oil-in-water emulsion, the emulsion comprising 2,6-diisopropylphenol dissolved in a water-immiscible solvent, emulsified with water and stabilized with a surfactant and wherein the oil-in-water emulsion further comprises cysteine or a salt thereof.

The present invention also relates to methods of administering 2,6-diisopropylphenol to a subject in need of anesthesia comprising parenterally delivering to the subject one of the above-mentioned sterile pharmaceutical compositions.

### DETAILED DESCRIPTION OF THE INVENTION

The present invention relates to pharmaceutical compositions comprising 2,6-diisopropylphenol (propofol) or a prodrug of propofol and cysteine. Compositions of the present invention comprise propofol, cysteine and one, two, three, four or more excipients. The compositions are chemically and physically stable over a wide range of environmental conditions. The compositions exhibit comparable or better stability to currently available commercial oil-in-water propofol emulsions, such as Diprivan®. The propofol containing compositions are preferably sterile and are parenterally administered to any animal, including humans.

The term “composition,” as used herein, refers to a mixture comprising propofol as an active ingredient and cysteine. The compositions can be aqueous or non-aqueous.

An “excipient,” as those terms are used herein, refers to a material contained in a composition other than the primary active ingredient (i.e., propofol) or water. Excipients or additives can be inert or can chemically or physically affect other composition components. Excipients may also have active properties of their own. Excipients can include, but are not limited to, surface active agents (e.g., surfactants, emulsifiers, detergents, binders and wetting agents), salts, polymers, solvents, antimicrobials, preservatives, fillers, diagnostic agents, sugars, alcohols, acids, bases, and buffers. The propofol compositions can further comprise active agents in addition to propofol such as, for example, anesthetic and/or antioxidative agents.

“Cysteine” as used herein, refers to cysteine and any salts thereof. For example, cysteine HCl is included within the definition of cysteine.

The term “substantially free,” as used herein, refers to compositions that contain the indicated component in only minor amounts, for example, as an impurity accompanying another component or as an impurity produced by a degradation process. Compositions that are substantially free of a component contain that component in a minimal concentration, for example, of less than about 3%, less than about 1%, preferably less than about 0.5%, more preferably less than about 0.1%, or even more preferably less than about 0.05% (w/v) such as less than about 0.01% (w/v).

The present invention is directed to propofol compositions comprising cysteine. Applicants have made the unexpected discovery that cysteine can be added to propofol containing compositions while still retaining composition stability. Cysteine functions to eliminate or inhibit microbial growth.

In some embodiments, compositions of the present invention comprise propofol, cysteine, and at least one, at least two, at least three, or at least four excipients. In one embodiment, propofol is present at a concentration of about 1 to about 25 milligrams per milliliter of composition, more than 1 mg/ml, more than 2 mg/ml, more than 3 mg/ml, more than 4 mg/ml, more than 5 mg/ml, more than 6 mg/ml, more than 7 mg/ml, more than 8 mg/ml, more than 9 mg/ml, more than 10 mg/ml, more than 11 mg/ml, more than 12 mg/ml, more than 13 mg/ml, more than 14 mg/ml, more than 15 mg/ml, more than 16 mg/ml, more than 17 mg/ml, more than 18 mg/ml, more than 19 mg/ml, more than 20 mg/ml, more than 21 mg/ml, more than 22 mg/ml, more than 23 mg/ml, more than 24 mg/ml, more than 25 mg/ml, more than 26 mg/ml, more than 27 mg/ml, more than 28 mg/ml, more than 29 mg/ml, more than 30 mg/ml, more than 31 mg/ml, more than 31 mg/ml, more than 32 mg/ml, more than 33 mg/ml, more than 34 mg/ml, more than 35 mg/ml, more than 36 mg/ml, more than 37 mg/ml, more than 38 mg/ml, more than 39 mg/ml, more than 40 mg/ml, more than 41 mg/ml, more than 42 mg/ml, more than 43 mg/ml, more than 44 mg/ml, more than 45 mg/ml, more than 46 mg/ml, more than 47 mg/ml, more than 48 mg/ml, more than 49 mg/ml, more than 50 mg/ml, more than 60 mg/ml, more than 70 mg/ml, more than 80 mg/ml, more than 90 mg/ml, or more than 100 mg/ml). Alternatively, between about 5 and about 20, about 5 and about 15, or about 8 and about 12 milligrams of propofol per milliliter of composition are present. Preferably, propofol is present at about 9 to about 11 milligrams per milliliter of composition, for example, about 10 mg/mL, or about 15 mg/ml, or about 20 mg/ml, or about 25 mg/ml. Alternatively, propofol compositions can be expressed as propofol percent weight/volume (w/v). For example, compositions of the invention can have propofol compositions of at least 0.5, 0.75, 1.0, 1.25, 1.5, 1.75, 2.0, 2.25, 2.5, 2.75, 3.0, 4, 5, 6, 7, 8, 9, 10, 11, 12, 13, 14, 15, 16, 17, 18, 19, 20, 21, 22, 23, 24, or 25 percent (w/v), or 0.5 to about 2.4, about 0.5 to about 2, about 0.5 to about 1.5, about 0.8 to about 1.2, or, preferably, about 0.9 to about 1.1 percent (w/v).

The present invention is directed to several propofol-containing compositions. In one embodiment, the compostitions are aqueous. Aqueous compositions of the instant invention can comprise propofol, cysteine, and one two, three, four, or more than four excipients, and water. For example, the excipients can be selected from the group consisting of ammonium acetate, poloxamer (e.g., Poloxamer 237 or Poloxamer 188), polyoxyethylene (23) lauryl ether (e.g., Brij® 35; Brij® is a trademark of ICI Americas, Inc.), polyoxyethylene (10) oleyl ether (e.g., Brij® 97), benzyl alcohol, polysorbate (e.g., polysorbate 20, i.e., polyethylene glycol sorbitan monolaurate (Tween® 20); or polysorbate 80, i.e., polyethylene 20 sorbitan monooleate (Tween® 80)), D-alpha-tocopheryl polyethylene glycol 1000 succinate (i.e., vitamin E TPGS), chlorobutanol, Cremophor® EL (i.e., Polyoxyl 35 Castor Oil; Cremophor® is a trademark of BASF), polyoxyethylene stearate, propylene glycol, deoxycholate (e.g., sodium deoxycholate), diethanolamine, ethanol, glycerin, lactobionic acid, lysine acid, magnesium chloride, polyethylene glycol stearate (e.g., polyethylene glycol 40 stearate, also referred to herein as PEG-40 stearate), and polyethylene glycol (e.g., polyethylene glycol 400, also referred to herein as PEG-400). Any known excipient may be specifically included in the present invention, including the excipients disclosed in Handbook of Pharmaceutical Additives compiled by Michael and Irene Ash, Gower Publishing, 1995 (incorporated herein by reference in its entirety).

In another embodiment, composition of this invention are non-aqueous. Non-aqueous compositions can comprise propofol, cysteine, and one, two, three or more excipients. Excipients can be selected from water immiscible solvents such as 1) vegetable oil (examples include soy bean, safflower, cottonseed, corn, sunflower, arachis, castor or olive oil), 2) an ester of a medium or long-chain fatty acid, or 3) a palmitate, a glyceral ester or polyoxyl hydrogenated castor oil. Excipients can also be selected from surfactants such as non-ionic surfactants, ethoxylated ethers, polypropylene-polyethylene block co-polymers, phosphatides, egg phosphatide, and soy phosphatide. Excipients can also include tonicity modifiers such as glycerin. Other suitable excipients include ascorbic acid and gentisic acid and salts thereof and monosodium glutamate.

In some embodiments, the excipient or combination of two, three, four, or more than four excipients is present in the composition in a total concentration of about 1 to about 50%, about 2 to 30%, about 2 to 20%, about 2 to 15%, or about 2 to 10% (w/v), for example, about 8%, less than 40%, less than 30%, less than 29%, less than 28%, less than 27%, less than 26%, less than 25%, less than 24%, less than 23%, less than 22%, less than 21%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, or less than 3% (w/v).

Included as embodiments of the present invention are compositions or formulations that exclude a specified excipient. Any known excipient, including those disclosed herein or disclosed in Handbook of Pharmaceutical Additives (sic), may be specifically excluded from the present invention. Any one or more than one species of excipients may be excluded from the present invention. For e.g., D-alpha-tocopheryl polyethylene glycol 1000 succinate may be excluded from the present invention. Compositions or formulations that comprise a specific excipient exceeding a specified amount may also be excluded. For example, a composition or formulation comprising a specified excipient(s) with a concentration of 90% or more, 80% or more, 70% or more, 60% or more, 50% or more, 40% or more, 30% or more, 29% or more, 28% or more, 27% or more, 26% or more, 25% or more, 24% or more, 23% or more, 22% or more, 21% or more, 20% or more, 19% or more, 18% or more, 17% or more, 16% or more, 15% or more, 14% or more, 13% or more, 12% or more, 11% or more, 10% or more, 9% or more, 8% or more, 7% or more, 6% or more, 5% or more, 4% or more, 3% or more, 2% or more, or 1% or more (w/v) may be specifically excluded from the present invention. For example, the following may be specifically excluded from the present invention: 8% or more, or 10% or more of D-alpha-tocopheryl polyethylene glycol 1000 succinate (w/v); 10% or more or 20% or more of 2-hydroxypropyl-beta-cyclodextrin (w/v); 5% or more, or 30% or more of N-methylpyrrolidone or 2-pyrrolidone, 30% or more of propylene glycol (w/v); combination of either N-methylpyrrolidone or 2-pyrrolidone, and propylene glycol (or a combination of all three), wherein the combined concentration is 60% or more (w/v); 2.5% or more, or 5% or more of a bile acid salt (e.g., sodium glycocolate/glycocolic acid), 4% or more, or 7% or more of lecithin (e.g., soybean or egg), or a combined concentration of 5% or more, or 7.5% or more, or 10% or more of both a bile salt and a lecithin (w/v); 0.5% or more, or 1% or more of benzyl alcolhol (w/v); 5% or more, or 15% or more of polyethoxylated castor oil (w/v); 5% or more, 7.5% or more, or 10% or more of a cyclodextrin, such as a sulfoalkyl ether cyclodextrin or sulfobuty ether cyclodextrin. Classes of excipients may also be specifically included or excluded as a component of a composition or formulation of the present invention, and optionally including the concentrations.

Compositions of the present invention comprise cysteine or a salt thereof in a concentration sufficient to exhibit antimicrobial activity against those microorganisms most likely to contaminate the propofol compositions.

The compositions of the present invention preferably have a physiologically neutral pH, such as between about 5 and about 9. Although, compositions of this invention are not limited to any particular pH range. In some embodiments, the compositions can have a pH range of between 2 and 12, between 4 and 10, between 4 and 9, between 4 and 6, between 5 and 7, or between 5 and 6. The pH of the propofol containing compositions can be adjusted as necessary by, for example, the addition of a base or a salt thereof, for example, an alkali such as sodium hydroxide, potassium hydroxide, or the like. Alternatively, an acid or a salt thereof such as hydrochloric acid, citric acid, or the like can be used to adjust the pH of the compositions. The term “pH modifier,” as used herein, refers to substances such as acids, bases, or salts thereof that are used to adjust the pH of a composition. Methods for selecting substances for modification of pH are well known to those skilled in the art. One type of non-aqueous propofol composition is an oil-in-water emulsion. Typically, propofol containing oil-in-water emulsions, e.g., Diprivan®, are formulated at a pH of 6 to 9 to assure stabilization of the small oil particles contained therein. Applicants have discovered stable oil-in-water emulsions comprising cysteine that have pH of about 5.5 to about 6. Further, these oil-in-water emulsions comprising cysteine, or a salt thereof, exhibit antimicrobial activity at least comparable to that of the commercial EDTA-containing Diprivan® formulation.

Emulsion physical stability and clinical performance depend critically on the particle-size distribution of the formulation. While many currently used preservatives have the tendency to destabilize the oil-in-water emulsion through electrostatic interactions and thus compromise the stability of the particle size distribution, the compositions of the present invention exhibit stability of the particle size distribution even under stressed environmental conditions. In addition, these cysteine/cysteinate containing propofol compositions substantially prevent the growth of microorganisms for at least about 24 hours following adventitious, extrinsic contamination.

Propofol emulsions, composed of lipids, glycerol, and large amounts of water in an isotonic environment with neutral to alkaline pH, provide a medium quite conducive to the growth of many microorganisms. As such, these oil-in-water emulsions require stringent handling, administration, and storage requirements. In addition, oil-in-water propofol emulsions typically require the presence of at least one preservative or antimicrobial. In one embodiment, the compositions are substantially microorganism-free pharmaceutical compositions, in particular, sterile pharmaceutical compositions. Preferably, the compositions are sterile and pyrogen-free.

One embodiment comprises propofol and cysteine. A further embodiment comprises propofol, cysteine, and one or more excipeints.

Another embodiment includes a sterile pharmaceutical composition for parenteral administration which comprises an aqueous solution of propofol, and which further comprises cysteine, and wherein said aqueous propofol solution is sufficient to prevent no more than a 10-fold increase in growth, or will support no more than a 10-fold increase in growth, of each of Staphylococcus aureus ATCC 6538, Escherichia coli ATCC 8739, Pseudomonas aeruginosa ATCC 9027 and Candida albicans ATCC 10231 for at least 24 hours as measured by a test wherein a washed suspension of each said organism is added to a separate aliquot of said composition at approximately 50 colony forming units per ml, at a temperature in the range 20° C. to 25° C., whereafter said aliquots are incubated at 20° C. to 25° C. for 24 hours and thereafter tested for viable counts of said organism. Another embodiment includes a method for producing anaesthesia in a warm-blooded animal which comprises parenterally administering to said animal in need thereof an anaesthetically effective amount of a sterile pharmaceutical composition which comprises an aqueous solution of propofol, and which further comprises cysteine wherein said aqueous propofol solution is sufficient to prevent no more than a 10-fold increase in growth, or will support no more than a 10-fold increase in growth, of each of Staphylococcus aureus ATCC 6538, Escherichia coli ATCC 8739, Pseudomonas aeruginosa ATCC 9027 and Candida albicans ATCC 10231 for at least 24 hours as measured by a test wherein a washed suspension of each said organism is added to a separate aliquot of said composition at approximately 50 colony forming units per ml, at a temperature in the range 20° C. to 25° C., whereafter said aliquots are incubated at 20° C. to 25° C. for 24 hours and thereafter tested for viable counts of said organism.

In one embodiment, the compositions of this invention comprise propofol, an emulsifier, a surfactant, a tonicity modifier and cysteine.

In another embodiment, the compositions of the invention comprise propofol at about 0.5 to 10%, at about 0.5 to 5%, at about 0.5 to 2%, at about 0.5 to 1.5%, at about 1%, at about 1.5 to 3%, or at about 1 to 2% (w/v); soybean oil at about 2 to 25%, at about 3 to 15%, at about 5 to 15%, at about 8 to 12%, or at about 10% (w/v); glycerol at about 0.5 to 10%, at about 0.5 to 5%, at about 1 to 4%, at about 1.5 to 3%, or at about 2% (w/v); egg lecithin at 0.5 to 5%, at about 0.5 to 4%, at about 1 to 4%, at about 1.5 to 3%, at about 2 to 5%, or at about 1 to 2% (w/v); and cysteine at about 0.2 to 5%, at about 0.5 to 4%, at about 0.5 to 2%, at about 1 to 2%, or at about 1% (w/v).

In another embodiment, the compositions of the invention comprise propofol at 1% w/v, soybean oil at 10% w/v, glycerol at 2.25% w/v, egg lecithin at 1.2% w/v and cysteine at 1% w/v.

Another embodiment comprises propofol and a preservative. Preservatives can be selected from cysteine, sodium ascorbate, gentisic acid, and monosodium glutamate.

In another embodiment, the compositions of the invention comprise propofol at about 0.5 to 10%, at about 0.5 to 5%, at about 0.5 to 2%, at about 0.5 to 1.5%, at about 1%, at about 1.5 to 3%, or at about 1 to 2% (w/v); soybean oil at about 2 to 25%, at about 3 to 15%, at about 5 to 15%, at about 8 to 12%, or at about 10% (w/v); glycerol at about 0.5 to 10%, at about 0.5 to 5%, at about 1 to 4%, at about 1.5 to 3%, or at about 2% (w/v); egg lecithin at 0.5 to 5%, at about 0.5 to 4%, at about 1 to 4%, at about 1.5 to 3%, at about 2 to 5%, or at about 1 to 2%(w/v); and sodium ascorbate at about 1 to 10%, at about 2 to 8%, at about 2 to 6%, at about 3 to 5%, or at about 4% (w/v).

In an additional embodiment, the compositions of the invention comprise propofol at about 0.5 to 10%, at about 0.5 to 5%, at about 0.5 to 2%, at about 0.5 to 1.5%, at about 1%, at about 1.5 to 3%, or at about 1 to 2% (w/v); soybean oil at about 2 to 25%, at about 3 to 1.5%, at about 5 to 15%, at about 8 to 12%, or at about 10% (w/v); glycerol at about 0.5 to 10%, at about 0.5 to 5%, at about 1 to 4%, at about 1.5 to 3%, or at about 2% (w/v); egg lecithin at 0.5 to 5%, at about 0.5 to 4%, at about 1 to 4%, at about 1.5 to 3%, at about 2 to 5%, or at about 1 to 2%(w/v); and gentisic acid at about 0.002 to 1%, at about 0.01 to 1%, at about 0.01 to 0.05%, at about 0.015 to 0.025%, or at about 0.02% (w/v).

In another embodiment, the compositions of the invention comprise propofol at about 0.5 to 10%, at about 0.5 to 5%, at about 0.5 to 2%, at about 0.5 to 1.5%, at about 1%, at about 1.5 to 3%, or at about 1 to 2% (w/v); soybean oil at about 2 to 25%, at about 3 to 15%, at about 5 to 15%, at about 8 to 12%, or at about 10% (w/v); glycerol at about 0.5 to 10%, at about 0.5 to 5%, at about 1 to 4%, at about 1.5 to 3%, or at about 2% (w/v); egg lecithin at 0.5 to 5%, at about 0.5 to 4%, at about 1 to 4%, at about 1.5 to 3%, at about 2 to 5%, or at about 1 to 2%(w/v); and monosodium glutamate at about 0.02 to 2%, at about 0.05 to 1%, at about 0.05 to 0.5%, at about 0.05 to 0.15%, or at about 0.1% (w/v).

Pharmaceutical compositions that are intended for application to delicate membranes of the body are commonly adjusted to approximately the same tonicity (i.e., isotonicity) as that of the body fluids. Isotonic compositions are those that cause minimal swelling or contraction of tissues upon contact, and produce little or no discomfort when instilled in body tissues. Preferably, the propofol compositions are substantially isotonic. The compositions may additionally comprise one or more tonicity modifiers. Examples of tonicity modifiers include, but are not limited to, lactose, dextrose, dextrose anhydrous, mannitol, sodium chloride, potassium chloride, propylene glycol and glycerin. Tonicity modifiers can be present in the compositions in concentrations of less than about 40, 30, 20, 10, or less than about 8 percent (w/v), e.g., about 0.5 to about 6, about 1 to about 3, about 2 to about 2.5, or about 2.3 percent (w/v).

In some embodiments, compositions of this invention can comprise cystine, or a salt thereof, instead of or in addition to cysteine. Isomers, both levorotatory and dextorotatory, of both cysteine and cystine are included within this invention.

Compositions of the invention may also include a prodrug of propofol.

The propofol containing compositions are preferably provided or administered as sterile pharmaceutical compositions. For example, the propofol containing compositions are administered substantially free of microorganisms. The preparation of sterile pharmaceutical compositions is well known to those experienced in the art. Sterile propofol containing compositions can be prepared using conventional techniques such as, for example, sterilization of final products or aseptic manufacture. In a preferred embodiment, the sterile compositions of the invention are substantially free of microorganisms for a longer period of time after opening than currently available propofol compositions such as Diprivan® Injectable Emulsion.

The compositions of the present invention can be provided in forms that possess desired propofol concentrations and are ready for direct administration to a patient. Alternatively, compositions can be provided in a concentrated form that requires dilution, for example, with water or an injectable solution, prior to administration. In the case of intravenous administration, the compositions can be admixed with diluents suitable for intravenous administration well known to those experienced in the art. Such diluents include water and injectable, aqueous sodium chloride and dextrose solutions.

The water used in the compositions of the present invention is preferably suitable for animal, including human, injection. The water should meet appropriate government and/or health care industry standards. Preferably, the water meets United States Pharmacopeia (USP) 23 standards for Pharmaceutical Grade Water for Injection. Normally, the water should contain no added substances.

Manufacture of emulsions may be performed by any of the various methods known in the art. An emulsification process may be batch or continuous. Examples of suitable apparatus for mixing components include jet mixers, injectors, mixing nozzles, pumps, agitated line mixers, packed tubes, gas agitated vessels, and stirred vessels, among others. Production of emulsions is well known to those of ordinary skill in the art and may be preformed without undue experimentation. Optionally, compositions of the present invention can be filtered to produce compositions comprising particles of desired sizes or size distributions. Methods for filtering such compositions are also well known to those skilled in the art.

Manufacturing aqueous compositions of this invention are known in the art. Simple mixing of the propofol and excipients is often sufficient.

The compositions of the invention can be characterized by the chemical stability of the therapeutic, prophylactic or diagnostic agents, e.g., propofol, that comprise the particles. The chemical stability of a constituent anesthetic agent can affect important characteristics of a pharmaceutical composition including shelf life, proper storage conditions, acceptable environments for administration, biological compatibility, and effectiveness of the agent. Chemical stability can be assessed using techniques well known in the art. For example, assays to detect degradation information obtained from stress studies (e.g., products of acid and base hydrolysis, thermal degradation, photolysis, and oxidation) for both active ingredients and excipients are numerous. One example of a technique that can be used to assess chemical stability is reverse phase high performance liquid chromatography (HPLC).

The compositions of the invention do not exhibit substantial propofol degradation such as, for example, no more than about 5% or no more than about 3% loss of propofol potency at room temperature over a given study period. Alternatively, propofol degradation can be assessed by measuring propofol degradate concentrations such as, for example, quinone and dimer concentrations. In some embodiments, the compositions do not exhibit substantial increases in propofol degradates such as, for example, no more than about 0.05%, no more than about 0.1%, or no more than about 0.2% increase in propofol degradate concentration over a given study period. In a preferred embodiment, any single degradate does not exceed the International Conference on Harmonization (ICH) guidelines, unless specific qualification of that degradate has been performed. (See ICH Document Q3B).

In one embodiment, the compositions do not experience substantial propofol degradation for a period of at least about 6 months when stored refrigerated. Preferably, the compositions do not experience substantial propofol degradation for a period of at least about one year when stored refrigerated. Even more preferred, the compositions do not experience substantial propofol degradation for at least about 6 months, for at least about one year, or, most preferably, for at least about two years when stored at or below about room temperature.

The compositions can be provided, prepared, stored, or transported in any container suitable for maintaining sterility. The container can incorporate means for dispensing composition such as, for example, a pierceable or removable seal. The compositions can be dispensed, for example, by extraction with a syringe or by pouring the composition directly into a device (e.g., a syringe, intravenous (IV) bag, or machine) for administration to a subject. Other means for providing, preparing, storing, transporting, and dispensing sterile pharmaceutical compositions are known to those skilled in the art.

In one embodiment, the compositions of the invention are manufactured, packaged, stored, or administered under an oxygen free atmosphere since 2,6-diisopropylphenol is subject to oxidative degradation. Oxygen free atmospheres include nitrogen, argon, or krypton gas, among others. Preferably, the compositions are manufactured, packaged, and stored under a nitrogen gas atmosphere.

The present invention is also directed to methods of administering 2,6-diisopropylphenol to a subject in need of anesthesia, the methods comprising intravenously delivering to the subject a sterile pharmaceutical composition. Sterile pharmaceutical compositions acceptable for delivery to a subject are described herein.

The compositions of the present invention can be administered to a subject for the induction and/or maintenance of anesthesia. The compositions can be parenterally administered to any animal, in particular, humans. In one embodiment, administration of a propofol containing composition comprises delivering the composition to a subject as a sole anesthetic, for example, via a bolus injection. In another aspect, administration of a propofol containing composition comprises delivering the composition to a subject for the induction of anesthesia and subsequently maintaining anesthesia with another anesthetic. Alternatively, administration of a propofol containing composition comprises delivering the composition to a subject for the induction and maintenance of longer-term anesthesia, for example, via continuous infusion. The compositions also can be delivered to a subject via intramuscular (i.e., IM) means, e.g., IM injection of propofol for induction and/or maintenance of anesthesia or intrathecal.

The propofol compositions can comprise active agents in addition to propofol or, alternatively, the propofol compositions can be co-administered with compositions comprising additional active agents. For example, the propofol containing compositions can comprise or be co-administered with one or more local anesthetic agents to reduce or eliminate injection pain. If administered, local anesthetic agents preferably are administered in concentrations sufficient to reduce or eliminate injection pain. Lidocaine is one example of a local anesthetic suitable for use in the instant compositions. One of ordinary skill in the art can select and administer concentrations of local anesthetic agent(s) to achieve the desired effects without undue experimentation.

The propofol containing compositions can be administered to a patient using techniques commonly known in the art. For example, the compositions can be delivered intravenously to a subject via bolus injection or by infusion. Infusion of the propofol containing compositions can be made by directly infusing a composition or, alternatively, by addition of a propofol containing composition to an appropriate infusion solution such as 0.9% sodium chloride injection, 5% dextrose injection, or another compatible infusion solution.

The quantity of propofol delivered to a subject during administration can be varied, as determined appropriate, by the physician supervising the administration.

The present invention includes a method of delivering propofol to a subject in need of anesthesia, the method comprising administering to a human or veterinary patient the sterile aqueous pharmaceutical composition described above.

The invention is further illustrated by the following non-limiting exemplification. The contents of all the references cited throughout this application are expressly incorporated herein by reference.

### EXEMPLIFICATION

### Example 1

This example describes the procedure used for producing sterile placebo emulsion formulations of propofol. In a sterile hood, the pH of 100 ml sterile Intralipid® emulsion was measured using a pH meter (pH ˜8.0) and adjusted to the pH indicated in Table 1 using 1 N NaOH. (Intralipid® is an oil-in-water nutritional injectable emulsion composition identical to Diprivan® except that it contains no preservatives and no propofol.) Upon stirring with a magnetic stirrer, a mass of preservative indicated in Table 1 was added to the Intralipid® emulsion (for example, in the case of cysteinate HCl, 0.15 g cysteinate HCl powder was added to the pH 10 Intralipid® emulsion). The pH of the resulting emulsion was then measured and titrated to pH 5.5 using 1 N NaOH or 1 N HCl. The emulsion was then filtered through a 0.45 m syringe filter into two 30 ml sterile vials (Hollister-Stier Laboratories, Spokane, Wash.).

TABLE 1
Preservatives contained in sterile placebo
emulsion formulations of propofol

| | | | pH of Intralipid ® |
|---|---|---|---|
| | | | emulsion |
| | Concentration | Weight | (prior to addition of |
| Preservative | (% w/v) | (g) | preservatives) |
| Cysteine HCl | 1 | 0.15 | 10.0 |
| Sodium ascorbate | 4 | 4.0 | 8.0 |
| Gentisic acid | 0.02 | 0.02 | 8.0 |
| Monosodium glutamate | 0.1 | 0.1 | 8.0 |

Emulsions thus formed were then subjected to stressed environmental conditions including shaking and freeze-thaw cycles. Compositions having pH of 4.5, 5.5, and 6.5 were prepared for these studies. Each of these emulsion formulations exhibited stability of particle size distribution comparable to that of a control Intralipid® emulsion.

### Example 2

Sterile placebo emulsion formulations of propofol were prepared as in Example 1. The growth retarding capability of these 4 placebo injectable emsulsions (each emulsion composition containing one of sodium ascorbate, cysteine/cysteine HCl, gentisic acid, and monosodium glutamate) were evaluated using membrane filtration technique and broth cultures. Approximately 200 colony forming units (CFU) per mL of four standard organisms recommended by United States Pharmacopeia (USP) for preservative efficacy tests were inoculated in each formulation. These four organisms are identified as Staphylococcus aureus (ATCC 6538), Escherichia coli (ATCC 8739), Pseudomonas aeruginosa (ATCC 9027), and Candida albicans (ATCC 10231). Microbiological testing of the prepared compositions was performed by Lancaster Laboratories (Lancaster, Pa.).

The antimicrobial activity of Intralipid® emulsion containing the 4 different preservatives were compared with two commercial propofol formulations, Diprivan® (AstraZeneca), which is a propofol formulation containing 0.005% disodium ethylenediaminetetraacetic acid, and a generic propofol formulation (Gensia Sincor) containing 0.025% sodium metabifulfite, as well as with a positive control (i.e., a control Intralipid® formulation lacking preservative). After the inoculation of the test organisms, test formulations were incubated at 30° C. The viable count of the test organism was determined immediately following the inoculation and after 24 hours and 48 hours of incubation at 30° C. The samples of the 4 preservative-containing Intralipid® formulations were from two freshly prepared 30-mL sterile vials. The Diprivan samples were from two fresh 50-mL syringes. Generic propofol samples were from two fresh 25-mL vials. Unpreserved Intralipid® formulation samples contained the same ingredients as those of the 4 testing formulations, except that they contained no preservatives. The preservative was considered effective if the microbial growth was suppressed, or allowed for a no-more-than 10-fold increase in growth as compared to the zero-hour viable count (i.e., the count of the organism immediately following inoculation) of each of the test organisms.

Tables 2 through 5 compare the antimicrobial effectiveness of the cysteinate HCl formulation with that of Diprivan® and generic propofol formulation, as well as unpreserved Intralipid®. These results indicate that cysteine/cysteinate HCl is competent to prevent the significant growth of microorganisms for at least 24 hours after adventitious, extrinsic contamination.

TABLE 2
Comparison of microbial growth retarding activity of
various formulations against S. aureus (ATCC 6538)

| | Visible count | | | Decrease in | Decrease in |
|---|---|---|---|---|---|
| | of survivors | | | survivors after | survivors after |
| | (Log₁₀ CFU/mL) | | | 24 h | 48 h |
| Formulation | 0 h | 24 h | 48 h | (Log CFU/mL) | (Log CFU/mL) |
| Intralipid ®/ | 2.3 | 1.9 | 1.8 | 0.4 | 0.5 |
| cysteine HCl | | | | | |
| Diprivan ® | 2.3 | 1.9 | 2.1 | 0.4 | 0.2 |
| (w/EDTA) | | | | | |
| Propofol emulsion | 2.3 | 2.0 | 0.0 | 0.3 | 2.3 |
| (w/metabisulfite) | | | | | |
| Unpreserved | 2.3 | 4.6 | 6.1 | NA | NA |
| Intralipid ® | | | | | |

TABLE 3
Comparison of microbial growth retarding activity of various
formulations against P. aeruginosa (ATCC 9027)

| | Visible count | | | Decrease in | Decrease in |
|---|---|---|---|---|---|
| | of survivors | | | survivors after | survivors after |
| | (Log₁₀ CFU/mL) | | | 24 h | 48 h |
| Formulation | 0 h | 24 h | 48 h | (Log CFU/mL) | (Log CFU/mL) |
| Intralipid ®/ | 2.2 | 0.6 | 0.0 | 1.6 | 2.2 |
| cysteine HCl | | | | | |
| Diprivan ® | 2.2 | 1.1 | 3.4 | 1.1 | NA |
| (w/EDTA) | | | | | |
| Propofol emulsion | 2.2 | 0.8 | <0.1 | 1.4 | 2.1 |
| (w/metabisulfite) | | | | | |
| Unpreserved | 2.2 | 1.6 | 3.7 | 0.6 | NA |
| Intralipid ® | | | | | |

TABLE 4
Comparison of microbial growth retarding activity of
various formulations against E. coli (ATCC 8739)

| | Visible count of survivors | | |
|---|---|---|---|
| | (Log₁₀ CFU/mL) | | |
| Formulation | 0 h | 24 h | 48 h |
| Intralipid ®/ | 2.4 | 3.2 | 4.2 |
| cysteine HCl | | | |
| Diprivan ® | 2.4 | 3.2 | 4.6 |
| (w/EDTA) | | | |
| Propofol emulsion | 2.4 | 2.7 | 2.7 |
| (w/metabisulfite) | | | |
| Unpreserved | 2.4 | 6.6 | 7.8 |
| Intralipid ® | | | |

TABLE 5
Comparison of microbial growth retarding activity of
various formulations against C. albicans (ATCC 10231)

| | Visible count of survivors | | |
|---|---|---|---|
| | (Log₁₀ CFU/mL) | | |
| Formulation | 0 h | 24 h | 48 h |
| Intralipid ®/ | 2.3 | 3.3 | 5.0 |
| cysteine HCl | | | |
| Diprivan ® | 2.3 | 2.4 | 2.3 |
| (w/EDTA) | | | |
| Propofol emulsion | 2.3 | 4.3 | 5.2 |
| (w/metabisulfite) | | | |
| Unpreserved | 2.3 | 5.3 | 7.0 |
| Intralipid ® | | | |

While this invention has been particularly shown and described with references to preferred embodiments thereof, it will be understood by those skilled in the art that various changes in form and details may be made therein without departing from the scope of the invention encompassed by the appended claims.

We claim:

1. A pharmaceutical composition comprising propofol and cysteine.

2. The pharmaceutical composition of claim 1, further comprising one or more excipients.

3. The pharmaceutical composition of claim 1, further comprising a GRAS excipient.

4. The pharmaceutical composition of claim 1, further comprising purified poloxamer, Ammonium acetate, Benzalkonium chloride, Benzethonium chloride, Benzyl alcohol, Brij 35, Brij 97, Calcium gluceptate, ChlorobutanOL, Citric Acid, Cremophor EL, Deoxycholate, Diethanolamine, Ethanol, Gamma cyclodextrin, Glycerin, Lactobionic acid, Lysine, Magnesium chloride, Methylparaben, PEG 1000, PEG 300, PEG 3350, PEG 400, PEG 600, Poloxamer 188, Poloxamer 237, Poloxamer 338, Poloxmer 407, Polyoxyethylene 100 stearate, Polyoxyethylene 40 stearate, Polyoxyethylene 50 stearate, Polysorbate 20, Polysorbate 80, Povidone, Propylene Glycol, Sodium acetate, Vitamin E TPGS, Sodium benzoate, Sodium tartate, vegetable oil, soy bean oil, safflower oil, cottonseed oil, corn oil, sunflower oil, arachis oil, castor oil, olive oil, an ester of a medium or long-chain fatty acid, a palmitate, a glyceral ester, polyoxyl hydrogenated castor oil, ethoxylated ethers, polypropylene-polyethylene block co-polymers, phosphatides, egg phosphatide, soy phosphatide, glycerin, ascorbic acid, gentisic acid, or monosodium glutamate.

5. The pharmaceutical composition of claim 1, wherein said composition is:
a. an aqueous solution; or
b. a non-aqueous solution.

6. The pharmaceutical composition of claim 1, wherein said cysteine is of sufficient concentration to allow a no more than 10-fold increase in growth of each of Staphylococcus aureus ATCC 6538, Escherichia coli ATCC 8739, Pseudomonas aeruginosa ATCC 9027 and Candida albicans ATCC 10231 for at least 24 hours as measured by a test wherein a washed suspension of each said organism is added to a separate aliquot of said composition at approximately 50 colony forming units per ml, at a temperature in the range 20° C. to 25° C., whereafter said aliquots are incubated at 20° C. to 25° C. for 24 hours and thereafter tested for viable counts of said organism.

7. The pharmaceutical composition of claim 1, wherein said composition is administered:
a. intraveneous;
b. intramuscular; or
c. intrathecal.

8. The pharmaceutical composition of claim 1, wherein the pH of said composition is:
a. between about 4.5 and about 9;
b. between about 5 and about 7;
c. between about 5 and about 6; or
d. between about 5.5 and about 6.

9. A method of treating a patient by administering the pharmaceutical composition of claim 1.

10. The pharmaceutical composition of claim 1, further comprising a local anesthetic.

11. A pharmaceutical composition, comprising:
(a) propofol;
(b) a water immiscible solvent;
(c) a surfactant; and
(d) cysteine or a salt thereof.

12. The pharmaceutical composition of claim 11, further comprising:
a. a tonicity modifier;
b. glycerol; or
c. a pH modifier.

13. The pharmaceutical composition of claim 11, wherein said propofol is at a concentration of:
a. about 0.5 to 2.5% w/v;
b. about 0.5 to 1.5% w/v;
c. about 0.9 to 0.1% w/v; or
d. about 1% w/v.

14. The pharmaceutical composition of claim 11, wherein said water immiscible solvent is:
a. selected from the group consisting of:
i) soybean oil;
ii) vegetable oil; and
iii) a medium or long-chain fatty acid; and
b. present at a concentration of:
i) from about 5 to about 15% w/v;
i) from about 8 to about 12% w/v;
i) from about 9 to about 11% w/v; or
i) about 10% w/v.

15. The pharmaceutical composition of claim 11, wherein said surfactant is:
a. selected from the group consisting of:
i) polypropylene-polyethylene block co-polymers;
ii) egg phosphatide; and
iii) soy phosphatide; and
b. present at a concentration of:
i) from about 0.5 to about 5% w/v;
ii) from about 0.5 to about 2% w/v;
iii) from about 1 to about 1.5% w/v; or
iv) about 1.2% w/v.

16. The pharmaceutical composition of claim 11, wherein said cysteine is:
a. cysteine; or a salt thereof; and
b. present at a concentration of:
i) from about 0.5 to about 5% w/v;
ii) from about 0.5 to about 2% w/v;
iii) from about 0.9 to about 1.5% w/v; or
iv) about 1% w/v.

17. A pharmaceutical composition comprising propofol, one or more excipients, and a preservative selected from the list consisting of:
a. sodium ascorbate;
b. gentisic acid; and
c. monosodium glutamate.
